(19) United States
(12) Patent Application Publication
(10) Pub. No.: US 2012/0289799 A1
(43) Pub. Date: Nov. 15, 2012

(54) High-Throughput Alignment-Insensitive Optical Connector for Laser-Based Photoplethysmography

(75) Inventors: Theodore Philip Delianides, Boulder, CO (US); Jonas Alexander Pologe, Boulder, CO (US)

(73) Assignee: KESTREL LABS, INC., Boulder, CO (US)

(21) Appl. No.: 13/107,226

(22) Filed: May 13, 2011

(51) Int. Cl. A61B 5/1455

(52) U.S. Cl. 600/322

(57) ABSTRACT

One embodiment of an optical coupling for use in a photoplethysmographic device having a first light guide (160) with core (150) that mates to a second light guide (210) that has a core (220) with a diameter and numerical aperture that are larger than those of the first light guide. This results in a high-throughput optical coupling that is insensitive to lateral offset and angular misalignments of the light guide axes. It allows the use of lower-cost, lower-precision molded parts in the manufacture of optical connectors, yet maximizes the light that is available for delivery to the tissue-under-test. Other embodiments are described and shown.

### STATEMENT REGARDING FEDERALLY SPONSORED RESEARCH OR DEVELOPMENT

This invention was made with government support under R44 HL073518 awarded by the National Institutes of Health. The government has certain rights in the invention.

### BACKGROUND-PRIOR ART

| U.S. Patents | | | |
|---|---|---|---|
| Pat. No. | Kind Code | Issue Date | Patentee |
| 7,313,427 | | Dec. 25, 2007 | Benni |
| 6,049,727 | | Apr. 11, 2000 | Crothall |
| 5,790,729 | | Aug. 4, 1998 | Pologe |

| Application | | Publication | |
|---|---|---|---|
| Number | Kind Code | Date | Applicant |
| US 2008/0071154 | A1 | Mar. 20, 2008 | Hausmann |

### BACKGROUND OF THE INVENTION

In the science of photoplethysmography, light is used to illuminate or trans-illuminate living tissue for the purpose of providing noninvasive measurements of blood analytes or other hemodynamic parameters or tissue properties. In this monitoring modality light is directed into living tissue and a portion of the light which is not absorbed by the tissues, or scattered in some other direction, is detected a short distance from the point at which the light entered the tissue. The detected light is converted into electronic signals that are indicative of the received light intensity exiting the tissue. These signals, one for each emitter, or spectral band of light incident on the living tissue (referred to in this specification as the tissue-under-test), vary with the pulsation of the blood through the tissue-under-test. These time-varying signals are referred to as photoplethysmographic signals. The photoplethysmographic signals are used to calculate blood analytes such as arterial blood oxygen saturation and/or hemodynamic variables such as heart rate, cardiac output, or tissue perfusion. Among the blood analytes that may be measured by photoplethysmography are various types of hemoglobin, including the percentages of oxyhemoglobin, carboxyhemoglobin, methemoglobin, and reduced hemoglobin in the arterial blood. A device which detects and processes photoplethysmographic signals to measure the levels of various blood analytes and/or various hemodynamic parameters is referred to as a photoplethysmographic measurement apparatus, photoplethysmographic device, or photoplethysmographic instrument.

The first widespread commercially-used photoplethysmographic device in medicine was the pulse oximeter, a photoplethysmographic device designed to measure arterial blood oxygen saturation. To measure oxygen saturation two different bands of light must be used, with each light band possessing a unique spectral content. Each spectral band, or light band, is typically referred to by a center wavelength, the centroid (or first moment of area of the wavelength distribution of the spectral band), or sometimes by a peak wavelength (the wavelength of maximum optical power). In conventional pulse oximetry two different emitters such as light emitting diodes (LEDs) are commonly used to generate the desired spectral bands. Usually one LED has a center, or peak, wavelength near 660 nanometers (nm) and a second LED has a center, or peak, wavelength near 900 nm. More recently photoplethysmographic instruments have been developed in which more than two light bands are utilized to allow the measurement of a larger number of blood analytes, including such blood analytes as oxyhemoglobin, carboxyhemoglobin, methemoglobin, and reduced hemoglobin.

Use of a photoplethysmographic instrument requires that light from each emitter (each light band) is incident on the tissue-under-test. On a person the tissue-under-test usually consists of a finger, earlobe, toe, foot, cheek, forehead, or other site on or, for invasive use, inside the body. The emitter light is delivered, via a sensor positioned on the tissue-under-test. The tissue-under-testis preferably well perfused with blood which helps provide a strong photoplethysmographic (or pulsatile) optical signal to be received at the detector that is typically also integral to the sensor. The detector is located a short distance from where the light enters the tissue-under-test, which allows for attenuation of the light signal by the pulsating blood flow within the tissue-under-test.

As the science of photoplethysmographic monitoring has progressed, an increasing number of light bands have been required to measure an increasing number of blood analytes. Furthermore, to improve the accuracy of measurement and the ability to discriminate between an ever-increasing number of blood analytes, it is most desirable to use spectrally-stable narrowband light sources. One type of spectrally-stable narrowband light source is a laser.

When using one or more lasers as light sources, or emitters, in a photoplethysmographic device, the lasers often cannot be placed in the sensor that is positioned in close proximity to, or directly on, the tissue-under-test, as has been typical with LED based photoplethysmographic sensors. This might be due to the physical size of the laser device being too large for placement in a small sensor designed for application to commonly-used sensing sites such as a finger. It also might be due to the need to position the laser in close proximity to its driver electronics, to one or more heat sinks, or to other electro-mechanical devices that, as a whole, create a package that is too large or cumbersome to place in the sensor or to conveniently position in immediate proximity to the tissue-under-test.

A typical photoplethysmographic instrument consists of a monitor, which provides the user interface for the instrument; a cable, which connects the monitor to a sensor; and the sensor, which is placed on the tissue-under-test. Many different but substantially equivalent configurations of the instrument are also possible. The lasers, given that they are not housed in the sensor, might be housed in the monitor or at some intermittent point along the cable connecting the monitor to the sensor. Regardless of exactly where the lasers are housed, as long as they are not at the sensor, the light emitted by the laser (or lasers) must be transmitted from the laser housing to the sensor, or at least to the sensing location on the tissue-under-test.

In such cases, this light transmission from the laser to the sensor is typically accomplished by employing a light guide. The light guide may be any one of a number of elements, or a chain of elements, including optical elements such as glass or plastic optical fibers, liquid filled light guides, fiber optic bundles, or other light pipes.

Light guides have been used in photoplethysmographic devices since the late 1970s when the first commercially available pulse oximeter went on the market. One early photoplethysmographic instrument used a pair of light guides in the form of two fiber bundles to both deliver the light, from a tungsten lamp source, to the tissue-under-test and to receive the light from the tissue-under-test and return the photoplethysmographic signals to the monitor for analysis. More recently, light guides have been used in pulse oximeters specifically designed for use on patients undergoing MRI (Magnetic Resonance Imaging) examination.

When trying to deliver light from one or more emitters to a tissue-under-test, it is often desirable to use a series of optical elements to deliver the light. For example, if the light sources, or emitters, are housed in the monitor and the light is transmitted down a cable to a sensor, it would be typical to have the sensor cable designed so that it could be removably attached to the monitor. This would allow easy replacement of the cable by the end user of the photoplethysmographic device, should the cable ever become damaged or nonfunctional.

Additionally it may be desirable to have the cable itself designed to be separable into two or more lengths or to be removably attached to the sensor. This would provide the ability to create an “extension cable” that would allow the monitor and the sensor to be separated by various different cable lengths as is sometimes required for varying clinical, applications. Furthermore, a cable providing a connector that can be separated at, or near, the sensor (the opto-mechanical and/or opto-electrical portion of the device that is positioned on the tissue-under-test) would make single patient use (or “disposable”) sensors practical. Clearly it could be prohibitively expensive to dispose of several meters of cabling each time a disposable sensor is thrown out.

If such an arrangement of one or more connectors is used to conduct light from the monitor to the sensor, the light must be conducted from the emitters, which might be located in the monitor housing, and then through a connector, down the cable, potentially through a second connector, and finally to the tissue-under-test.

Unfortunately, most optical connectors are designed to meet various needs of specific industries such as communications or defense, where low optical loss is only one of many important performance criteria. Additional requirements might include long service lifetime, durability, thermal stability, stringent core alignment tolerances and minimal numerical apertures to limit mode dispersion (which otherwise affects high bit-rate communications), and polarization orientation maintenance. The resulting high cost of such connectors, often additionally burdened with expensive cable termination and installation costs, makes existing optical connector technologies wholly impractical for commercially-viable pulse oximetry use, where low-cost cabling and sensors are the norm. The light transmitted to the tissue-under-test in pulse oximetry is purely for illumination purposes, and thus expensive single mode, low numerical aperture, or polarization-maintaining optical light guides and connectors are not required.

The cost of optical connectors for use in pulse oximetry are further impacted by the need to have multiple optical connections from multiple emitter sources in combination with multiple electrical conductors for photodetector and other electronic signals, all co-located in a single connector body for ease of use by the clinical end-user. Existing solutions for so-called “hybrid” multi-position electro-optical connectors can be extremely expensive and yet be very sensitive to dirt and moisture contamination that could create high optical losses that reduce the light available for measuring the desired blood analytes. The high cost of these hybrid connectors, and the problem of optical loss through such connectors, has severely limited the use of light guides, such as optical fibers or optical fiber bundles, in photoplethysmography to a few specialty applications such as the use of pulse oximetry in Magnetic Resonance Imaging (MRI) settings.

Exceedingly few optical-fiber-based photoplethysmographic instruments have been manufactured and marketed in the roughly three decades that commercial pulse oximetry has been in existence and none of these devices have found widespread use. The Minolta/Marquest Model SM-32 Oxygen Saturation Monitor was the first such instrument, predating even conventional “LED-based” oximeters. The Nonin Model 7500FO Fiber Optic Tabletop Pulse Oximeter, intended for MRI environments, is a much more recent introduction. Even though there are literally decades between the introductions of these two instruments, the light guide based sensor cables on both instruments are large, bulky, expensive, and impractical for widespread clinical use. In addition, both of these instruments provide only a single connector at the interface between the sensor and the monitor. Furthermore, because the light guides used in these instruments are relatively large fiber optic bundles that must be protected from damage during use, the resulting cables are bulky and have extremely limited flexibility. This lack of flexibility in the sensor cable creates additional problems by increasing the amount of artifactual signal obtained when there is patient motion at the sensor site, and the mere weight and stiffness of a bulky sensor cable can often cause the sensor to pull off the patient.

In addition to limiting the use of light guides in photoplethysmography to specialized applications, the problem of how to create low cost, compact, high optical throughput, hybrid electro-optical connectors has also made it highly impractical to use laser-based light sources in photoplethysmographic instruments.

U.S. Pat. No. 6,049,727 discusses a laser diode based apparatus designed to “determine the concentration of a constituent of the body fluid.” In this patent the light from a set of laser diodes is “coupled to an input of one single-mode optical fiber” which then transmits this light directly to the body fluid. Alternatively, this patent states that the light from each individual laser diode can be coupled into a series of individual optical fibers and then this series of optical fibers can be formed into a fiber bundle and coupled into a “single core multimode optical fiber 54 of approximately the same diameter as the fiber bundle 52.” This coupler, however, does not constitute a removable connection or connector. In fact, the patent does not disclose any form of connector located at any point along the single optical fiber through which, all the different wavelengths of light are transmitted from the instrument to the body fluid. Nor is the apparatus revealed in this patent designed to be a photoplethysmographic device.

The optical coupler described in U.S. Pat. No. 5,790,729 is, as with the couplers described in the previously-discussed patent, specifically designed to combine the light from multiple emitters into one or more optical channels (optical paths). The optical coupler is not used to create a connector for a sensor cable. While the patent discusses controlling the numerical aperture of the waveguide, the defined purpose is to “maximize the coupling [of the light sources into the waveguide].” Furthermore, the waveguide of this patent is not part of a connector and may not be practical for use in a removable connector. It therefore does not solve the problem of how to create a hybrid electro-optical cable connector solution that provides an inexpensive, low optical loss, removable connection of a sensor to a photoplethysmographic instrument.

U.S. Pat. No. 7,313,427 reveals an apparatus for combining “multiple laser light sources of different wavelengths to be coupled into a small diameter core fiber optic . . . ” In this apparatus a “combiner assembly” utilizes a ball lens arrangement to “focus the light” from several different optical fibers “onto a smaller diameter output fiber.” In doing this the patent states that the “numerical aperture ‘NA’ of the output fiber 116 is greater than or equal to [the NA of] the input fiber bundle.” The combiner of this patent is not designed for removable connection of one light guide to another but only to couple multiple fibers into a single fiber. Furthermore the use of a ball lens in the coupler design increases the cost of such a coupler by increasing the number of optical elements that must be held in alignment to one another, thereby making the overall optical alignment much more critical and increasing the mechanical constraints on the design of the coupler. Finally the coupler is defined as being intended for use in a Near InfraRed Spectrophotometric (NIRS) monitor, not a photoplethysmographic instrument.

US patent application publication No. 2008/0071154 discusses the use of light emitting nanostructure devices in spectrophotometric measurements, in general, and pulse oximetry, in specific. In this application a lens is used to match the numerical aperture of the light source to the “fiber diameter.” The application does not discuss or reveal any technique to match or optimize the numerical aperture of one light guide to another across a connector.

For the reasons described above it is desirable to create an inexpensive hybrid electro-optical connector, for coupling both electrical and optical signals, that: provides inexpensive coupling of the electrical and optical signals; imposes minimal optical losses on the optical signals; allows the use of thin light guides such as single optical fibers, if desired; and reduces the requirement for high-precision alignment of the opto-mechanical parts in the connector.

### BRIEF SUMMARY OF THE INVENTION

In accordance with one embodiment a low tolerance, high throughput optical connector for a photoplethysmographic device comprises two detachable parts each containing at least one light guide designed to transmit laser light from one light guide to the other, wherein the two light guides are coupled together by aligning them within a physical coupling or connector. Accordingly, several advantages of one or more aspects are as follows: that the optical power lost at the coupling due to lateral misalignment of the center axes of the two light guides can be minimized by using a light guide on the receiving side of the optical connector that has a larger diameter than that of the light guide on the transmitting side; that the optical power lost at the coupling due to angular misalignment of the central axes of the two light guides can be minimized by using a light guide on the receiving side of the optical connector that has a larger numerical aperture (NA) than that of the light guide on the transmitting side; that the combination of increasing NA and increasing light guide diameter, used individually or in combination, in the same connector help ensure that a maximum percentage of the laser light crossing the interface is coupled into the second light guide, thus maximizing the laser light available for sensing of the desired blood analytes, hemodynamic parameters, or tissue properties; and that the design constraints for this high throughput optical connector provide increased tolerance for mechanical misalignment, thus allowing increased imprecision of the connector in terms of mechanical design, molded part tooling, and assembly processes without reducing the optical performance of the connector.

### DRAWINGS

FIG. 1. Block diagram of a photoplethysmographic instrument with detachable cable and detachable sensor

FIG. 2. Light guide coupling between light guides of identical physical size but with different numerical aperture

FIG. 3. Light guide coupling between light guides of different physical size and numerical aperture

### DETAILED DESCRIPTION OF THE INVENTION

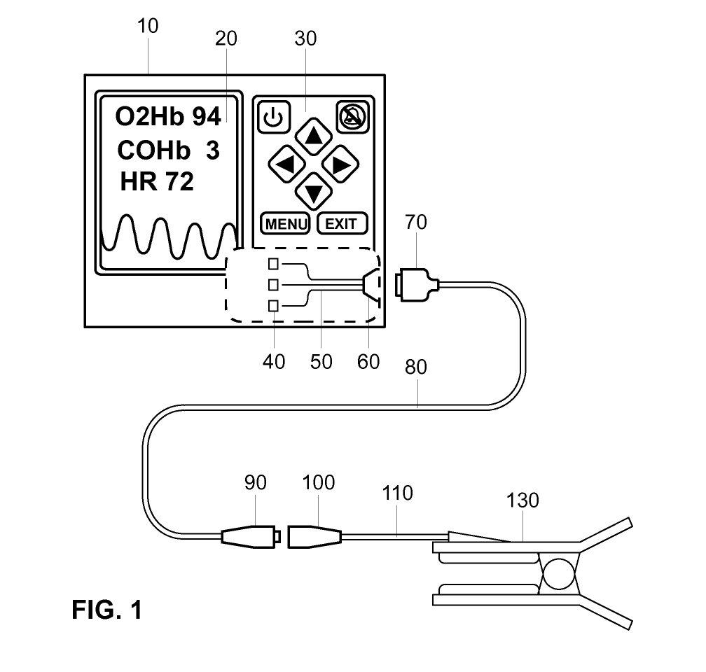

A block diagram of a photoplethysmographic instrument is shown in FIG. 1. A monitor 10 includes a display interface 20 which presents the monitored blood analytes or physiological parameters to the clinician or end user of the monitor. Monitor 10 also includes a user control panel 30 for controlling the operation of monitor 10. Also housed inside monitor 10 is at least one laser-based emitter 40 that emits light into a light guide 50. Note that in this block diagram three emitters 40 are shown, each coupled to an individual light guide 50. The light guide, or guides, 50 is arranged within a panel connector 60 such that it detachably mates to a second light guide (not shown) within a cable end connector 70. The second light guide continues down cable 80, which terminates at its distal end at connector 90. Connector 90 mates to connector 100 on the proximal end of cable 110, which attaches to a patent sensor 130. The light guide, or guides, within cable 80 mates to the light guide, or guides, within cable 110 via a detachable coupling between connectors 90 and 100. The cables 80 and 110, along with the connectors 60, 70, 90, and 100, could also include multiple electrical conductors (not shown) for the transmission of electrical signals such as the photodetector signals from the sensor 130.

In conventional photoplethysmographic devices the light sources, also called emitters, generate the light that is used for sensing the blood analytes or the physiological parameters to be measured. The analytes or physiological parameters to be measured may include arterial blood oxygen saturation or level (also referred to as O₂Hb, [O₂Hb], SaO₂, or SₚO₂), carboxyhemoglobin level (also referred to as COHb, [COHb], or SₚCO), methemoglobin level (also referred to as metHb, [metHb], or Sₚmet), pulse rate (also called heart rate, HR, or PR), and perfusion index (also called PI), along with, many others. In pulse oximetry, a common photoplethysmographic device, the emitters used for these measurements typically consist of light emitting diodes (LEDs), although several other light sources have been used including, in the earliest pulse oximeters, tungsten lamps.

In a conventional pulse oximeter the LEDs are housed in the sensor. Light emitted by the LEDs may pass through a diffuser, or other intervening optics, and then the light passes through an output window, or aperture, and is incident directly on the tissue-under-test. A small portion of the light then passes through the tissue-under-test and is received by a photodetector that is typically positioned a short distance from where the light originally entered the tissue-under-test. The photodetector signal is measured by the photoplethysmographic instrument and processed into the desired blood analyte measurements. The conventional pulse oximeter is only capable of measuring oxygen saturation (S_{P}O₂) and perhaps heart rate (HR) and perfusion index (PI).

With the increasing desire to measure more blood analytes and physiological parameters, and with ever-increasing accuracy, the emitter types now being used include lasers. Lasers are a type of emitter that can generate light with a much narrower spectral bandwidth than conventional LEDs. The use of lasers in photoplethysmographic devices provides the opportunity for increased measurement accuracy and precision as well as the opportunity to measure additional parameters and/or blood analytes that were not attainable with more broadband light sources.

The difficulty in using a laser light source is that the laser light must be transmitted from one location, such as the photoplethysmographic monitor, to a tissue-under-test, which means that a light guide system, consisting of a series of optical light guides, must be employed. The light guide (or a set of light guides, typically one for each emitter located some distance from the sensor) is typically contained within a cable that connects a monitor to a sensor located on a tissue-under-test. As discussed earlier, it is advantageous to have a means for disconnecting a cable and sensor at one or more locations, to facilitate replacement of worn parts, cleaning and disinfection, or the use of single-use (disposable) sensors.

In FIG. 1 connectors 60, 70, 90, and 100 provide detachable optical connections and might be called an instrument connector, cable connector, sensor connector, or other equivalent term. Within connector 60 is at least one transmitting light guide that is matched or aligned to transmit light to a receiving light guide in connector 70. Note that more than one pair of coupled light guides (also termed a series of light guides) could exist within the connectors 60 and 70. A similar arrangement of one or more series of light guides also exists between transmitting connector 90 and receiving connector 100 at the connection between the patient cable 80 and the sensor cable segment 110.

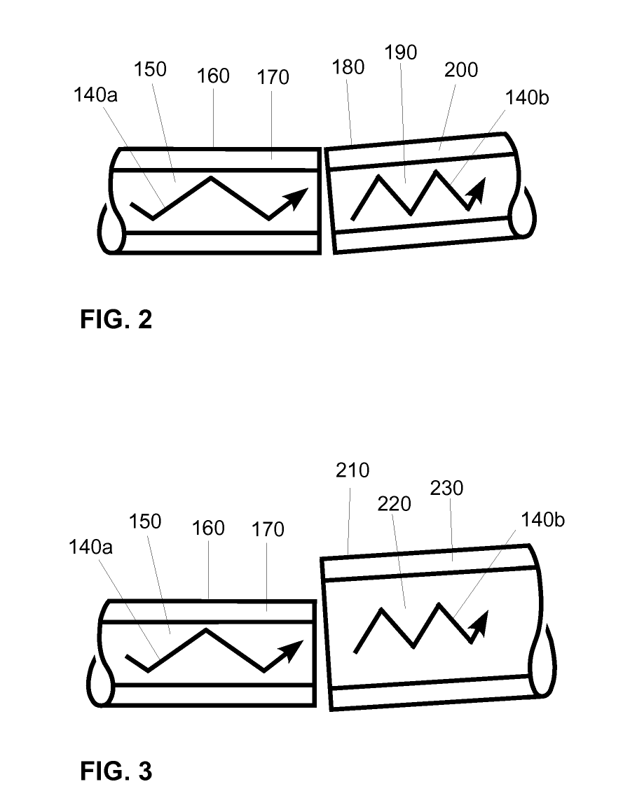

One embodiment of a high-throughput optical connection for laser-based photoplethysmography is shown in FIG. 2. It is a cross-sectional view of a coupling of a series of two light guides within a connector pair (such as 60 and 70 or 90 and 100 as shown in FIG. 1). The connector bodies have been omitted for clarity. A transmitting light guide 160 has a core 150 and a cladding 170, which are each typically defined in part by their respective outer diameter measurements if the cross section of the light guide is circular, although similar defining measurements for a core and cladding would apply to a square or other shape cross section of light guide. The transmitting light guide 160 also has a numerical aperture (NA) that allows the light guide core 150 to only accept and propagate a light ray 140a that travels through the core at an angle to the light guide axis that is at or below a specific limiting angle. This limiting angle is defined by the NA of the light guide and is related to the refractive indices of the core and cladding materials. The light guide axis could also be called the longitudinal axis of the light guide and could be defined as the axis that is coincident with the center axis of a substantially circular cross section light guide.

The transmitting light guide 160 and a receiving light guide 180 are held in alignment by the connector pair (60 and 70 or 90 and 100, not shown in FIG. 2) and thus are part of a detachable or removable optical connection. In the embodiment shown in FIG. 2, the receiving light guide 180 has a core 190 and a cladding 200 that are substantially the same physical sizes as the core and cladding, respectively, of light guide 160. The two light guide end faces are close enough together that they are considered to be in physical contact with each other, thus minimizing Fresnel reflection losses that would occur because of mismatches in refractive index (for example, as would occur between glass and air). The receiving light guide 180 has a larger NA that allows the propagation of a light ray 140b that can be at a steeper angle to the light guide axis than the allowable maximum angle inside the transmitting light guide 160 for light ray 140a.

In the embodiment shown in FIG. 2, if the two light guide axis end points, for the series of light guides 160 and 180, are in good lateral alignment at the adjacent light guide end faces, but there is a slight angular mismatch in their alignment as depicted in FIG. 2, the receiving light guide 180 is still able to receive most of the light transmitted from light guide 160, because it can accept and propagate light rays that are launched into its, core that are at a steeper angle to its longitudinal axis than compared to the case for the transmitting light guide. Such an arrangement minimizes the transmission loss, which can also be called an insertion loss, connector loss, or throughput loss across the mated connector pair. Optical losses due to angular misalignment of two light guides are well understood within the fiber optics industry and are one reason why optical connectors developed for applications such as communications and defense, where a light guide with the same physical size and NA must be used throughout the optical path, require a high precision, and consequently high cost, connection.

Another embodiment, also in cross-sectional view, is shown in FIG. 3. Again a first light guide 160 with a core 150 and a cladding 170 propagates light 140a towards the interface between the series of optical elements consisting of first light guide 160 and second light guide 210. Receiving light guide 210 has a core 220 that is larger in diameter than the core 150 of the transmitting light guide 160 and a cladding 230 that is larger in diameter than the cladding 170 of the transmitting light guide. The receiving light guide 210 also has a larger NA than that of the transmitting light guide 160, as depicted by the higher angle of light propagation 140b. In this embodiment, there is a lateral offset between the two light guide axes as well as an angular mismatch similar to FIG. 2; but, because the receiving light guide 210 has both a larger core 220 and a larger NA, very little light is lost at the interface between the light guides due to the combination of these two different types of misalignment. The smaller core 150 of transmitting light guide 160 is fully overlapped by the larger core 220 of receiving light guide 210, thus minimizing transmission losses across the connection. This arrangement also minimizes or eliminates losses due to an excessive core eccentricity or a lack of core/cladding concentricity, either of which can prevent proper overlap of the cores across the interface between the light guides, and both of which affect the performance of existing optical connectors. The use of a larger NA for the receiving light guide 210 minimizes the optical losses due to an angular mismatch between the light guides, as was explained for the embodiment in FIG. 2. The combination of the use of a larger diameter and a higher NA in light guide 210 versus light guide 160 creates an optical connection for a series of light guides for delivery of laser light from an emitter to a sensor in a photoplethysmographic device that is highly insensitive to multiple types of opto-mechanical misalignment.

While the embodiment shown in FIG. 3 illustrates aspects where the core diameter and NA of the receiving light guide are both larger than those of the transmitting light guide, the optical connector losses would be reduced using either of these aspects individually. Furthermore, these two aspects, whether used individually or in combination, allow for a high-throughput optical coupling that does not require precision alignment of the light guide axes. Such an optical coupling could be termed an “alignment-insensitive coupling”, and it allows the use of lower-precision molded parts that would otherwise not provide the necessary precision required when coupling light guides with identical physical sizes and/or numerical apertures. The ability to achieve such a low transmission loss optical connection in such a compact space further allows for integration of multiple light guide couplings co-located with one or more electrical couplings all within the same low-cost connector body.

The previous discussion of the embodiments has been presented for the purposes of illustration and description. The description is not intended to limit the invention to the form disclosed herein. Variations and modifications commensurate with the above are considered to be within the scope of the present invention. The embodiments described herein are further intended to explain the best modes presently known of practicing the invention and to enable others skilled in the art to utilize the invention as such, or in other embodiments, and with the particular modifications required by their particular application or uses of the invention. It is intended that the appended claims be construed to include alternative embodiments to the extent permitted by the prior art.

1) A light delivery apparatus for a photoplethysmographic device comprising:
a. a series of two or more optical elements;
b. the series of optical elements arranged to conduct light from a laser;
c. at least one of the optical elements comprised of a first light guide;
d. at least one of the optical elements comprised of a second light guide;
e. the first light guide and the second light guide arranged to conduct light from the first light guide to the second light guide in an alignment-insensitive connection.

2) The apparatus of claim 1 wherein the alignment-insensitive connection is provided by the second light guide having a larger core diameter than the first light guide.

3) The apparatus of claim 1 wherein the alignment-insensitive connection is provided by the second light guide having a larger numerical aperture than the first light guide.

4) A light delivery apparatus for a photoplethysmographic device comprising:
a. a series of two or more optical elements;
b. the series of optical elements arranged to conduct light from a laser;
c. at least one of the optical elements comprised of a first light guide;
d. at least one of the optical elements comprised of a second light guide;
e. the first light guide and the second light guide arranged to conduct light from the first light guide: to the second light guide;
f. the second light guide having a larger core diameter than the first light guide;
g. the second light guide further having a larger numerical aperture than the first light guide;
whereby coupling of light from the first light guide to the second light guide is highly insensitive to misalignment.

5) A method for delivering light in a photoplethysmographic measurement system comprising the steps of:
a. providing a plurality of optical elements including at least a first light guiding element and a second light guiding element;
b. arranging the optical elements to conduct light from a laser;
c. further arranging the optical elements such that light from the laser passes from the first light guiding element to the second light guiding element;
f. designing a connection between the first light guiding element and the second light guiding element to be alignment-insensitive;

6) The method of claim 5 wherein the designing step further comprises the step of selecting the second light guiding element to have a larger core than the first light guiding element.

7) The method of claim 5 wherein the designing step further comprises the step, of selecting the second light guiding element to have a larger numerical aperture than the first light guiding element.
